(19) United States
(12) Patent Application Publication
(10) Pub. No.: US 2019/0201326 A1
(43) Pub. Date: Jul. 4, 2019

(54) DRUG DELIVERY SYSTEMS AND TARGETED RELEASE OF PHARMACEUTICAL AGENTS WITH FOCUSED ULTRASOUND

(71) Applicant: THE JOHNS HOPKINS UNIVERSITY

(72) Inventors: Raag D. AIRAN, Washington, DC (US); Jordan J. GREEN, Nottingham, MD (US); Randall A. MEYER, Baltimore, MD (US)

(21) Appl. No.: 16/303,220

(22) PCT Filed: May 18, 2017
(86) PCT No.: PCT/US2017/033226

§ 371 Date: Nov. 20, 2018

Related U.S. Application Data
(60) Provisional application No. 62/339,176, filed on May 20, 2016.

(51) Int. Cl. A61K 9/00; A61K 9/16; A61K 31/05; A61M 37/00; A61K 41/00

(52) U.S. Cl. CPC A61K 9/0009; A61K 9/1641; A61K 31/05; A61M 2210/0693; A61K 9/1617; A61K 41/0028; A61M 37/0092

(57) ABSTRACT

The present invention is a new controlled drug system that can be used for targeting non-invasive neuromodulation enabled by focused ultrasound gated release of one or more small molecule neuromodulatory agents.

### REFERENCE TO RELATED APPLICATIONS

This application claims the benefit of U.S. Provisional Patent Application No. 62/339,176, filed on May 20, 2016, which is hereby incorporated by reference for all purposes as if fully set forth herein.

### BACKGROUND OF THE INVENTION

Controlled drug delivery systems (DDS) have several advantages compared to the traditional forms of drugs. A drug is transported to the place of action, hence, its influence on vital tissues and undesirable side effects can be minimized. Accumulation of therapeutic compounds in the target site increases and, consequently, the required doses of drugs are lower. This modern form of therapy is especially important when there is a discrepancy between the dose and the concentration of a drug and its therapeutic results or toxic effects. Cell-specific, or tissue targeting can be accomplished by attaching drugs to specially designed carriers. Various nanostructures, including liposomes, polymers, dendrimers, silicon or carbon materials, and magnetic nanoparticles, have been tested as carriers in drug delivery systems. There is a need to develop new controlled drug delivery systems for prevention or treatment of medical conditions.

### SUMMARY OF THE INVENTION

The present invention is a new controlled drug system that can be used for targeting non-invasive neuromodulation enabled by focused ultrasound gated release of one or more small molecule neuromodulatory agents.

One embodiment of the present invention is a nanoparticle with a surface comprising an expandable polymer encasing a pharmaceutical composition comprising a pharmaceutical agent and a material that expands upon the application of ultrasound. Any material able to transition from a liquid to solid when ultrasound is applied may be suitable for the present invention but the preferable expandable material is perfluoropentane. Suitable expandable polymers include block copolymers such as PEGylated poly-caprolactone, PEGylated poly-L-lactide, or a combination thereof, as examples. Most pharmaceutical agents may be suitable for the present invention but the preferred pharmaceutical agent is a neuromodulatory agent propofol.

Another embodiment of the present invention is a method of targeted release of a drug, or pharmaceutical agent, comprising the following steps: a) administering to a subject a nanoparticle with a surface comprising an expandable polymer encasing a pharmaceutical composition comprising a pharmaceutical agent and a material that expands upon the application of ultrasound; and b) applying ultrasound to an area of the subject adjacent to the nanoparticle so the material, expandable polymer, and surface expand forming an expanded surface that releases the pharmaceutical agent from the nanoparticle compared to when the ultrasound is not applied to the nanoparticle. Typically, applying the ultrasound to a nanoparticle of the present invention expands its diameter in the range of 5 to 6 times forming an expanded nanoparticle that releases one or more pharmaceutical agent(s) from the nanoparticle compared to when ultrasound is not applied to the nanoparticle. The ultrasound may be applied in many ways but it is preferred application is with a tip sonicator and/or a focused ultrasound transducer such as a MR-guided focused ultrasound system (MRgFUS). The ultrasound is preferably applied at 20 kHz continuously in the range of 1 to 10 seconds. However the ultrasound may be applied at 17 kHz, 18 kHz, 19 kHz, 21 kHz, 22 kHz, 23 kHz, 24 kHz, 25 kHz, 26 kHz, 27 kHz, 28 kHz, 29 kHz, 30 Khz, or in the range of 20 kHz to 25 kHz, 20 kHz to 30 kHz, or a range in between. The ultrasound may be applied for 1, 2, 3, 4, 5, 6, 7, 8, 9, 10, 11, 12, 13, 14, or 15 seconds. The ultrasound may be applied in the range of 2 to 9 second, 3 to 7 seconds, 4 to 6 seconds, or any range in between. Alternatively, the ultrasound may be applied at 1 mHz, 2 mHz, 3 mHz, 4 mHz, 5 mHz, or 6 mHz using 5 ms, 10 ms, 20 ms, 30 ms, 40 ms, 50 ms, 60 ms, 70 ms, 80 ms, 90 ms, 100 ms, or 110 ms pulses every 1 sec, 2 sec, 3 sec, 4 sec, 5 sec, 6 sec, 7 sec, 8 sec, 9 sec, or 10 sec for up to 1, 2, 3, 4, 5, 6, 7, 8, 9, or 10 minutes.

Another embodiment of the present invention is a brain functional localization method comprising the step of: a) administering to the brain of a subject a drug delivery system comprising a nanoparticle with a surface comprising an expandable polymer encasing a pharmaceutical composition comprising one or more neuromodulatory agent(s) and a material that expands upon the application of ultrasound; b) applying ultrasound to an area of the brain adjacent to the nanoparticle so the material, polymer, and surface expand forming an expanded surface that releases the neuromodulatory agent from the nanoparticle compared to when the ultrasound is not applied to the nanoparticle. Typically, applying the ultrasound to a nanoparticle of the present invention expands its diameter in the range of 5 to 6 times forming an expanded nanoparticle that releases one or more neuromodulatory agent(s) from the nanoparticle compared to when ultrasound is not applied to the nanoparticle. Most neuromodulatory agents are suitable for use in the present invention but the preferred neuromodulatory agent is propofol.

The term “MRgFUS” refers to a MR-guided ultrasound system.

The term “nanoparticle(s)” refers to a particle(s) having the size in the range of 100 nm to 400 nm, 300 nm to 600 nm, 320 nm to 580 nm, 340 nm to 560 nm, 360 nm to 540 nm, 380 nm to 520 nm, 400 nm to 500 nm, or 400 nm to 450 nm, for example.

The term “neuromodulatory” refers to neuromodulatory mechanisms that play an important role in allowing the nervous system to adapt to changes in context or behavioral state, and dysregulation of these mechanisms contributes to nervous system disorders.

The term “neuromodulatory agent” refers to an entity that affects neuromodulatory mechanisms such as the neuromodulation of synaptic function, behavioral state changes, neuromodulatory mechanisms to innate behavior and cognitive functions, and contributions of neuromodulatory mechanisms to disorders of the nervous system (as examples). An “entity” may be a neuropeptide, growth factor, a hormone, a chemical, a nucleic acid, an amino acid sequence, or a protein for example.

The term “sonication” refers to the act of applying sound energy to agitate particles in a sample, for various purposes.

The term “subject” refers to any individual or patient to which the method described herein is performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

The term “ultrasonic” or “ultrasound” refers to frequencies of greater than 17 kHz. The preferred ultrasonic frequency used in the present invention is 20 kHz.

### BRIEF DESCRIPTION OF THE DRAWINGS

FIG. 1 illustrates a schematic of some of the elements of the present invention.

FIG. 2 illustrates the schematic of nanoparticle design. Nanoparticles are preferably composed of a block copolymer (yellow and blue lines) encapsulating propofol (red dots) and a liquid perfluorocarbon droplet (light blue). Upon sonication, the liquid perfluorocarbon at the core of the particles transitions into a gas phase (lighter blue), triggering the release of propofol in this example.

FIG. 3A-3C illustrates in vitro sonication-induced release of propofol versus in situ pressure (left) and burst length (right) demonstrates a dose response with in situ pressure but not with burst length. Propofol release was assessed by UV fluorescence of the organic medium after hexane extraction of free propofol, post sonication using pulsed focused sonication with 1 MHz transducer frequency with 1 Hz burst frequency for 60 sec (60 total bursts). Sonication burst length of 100 ms was used for the left plot; and 1 MPa estimated in situ pressure was used for the right plot; n=3.

FIGS. 4A and 4B illustrates the biodistribution of nanoparticles of the present invention.

FIG. 5 illustrates a rat seizure model.

FIG. 6 illustrates sample EEG traces after in vivo intravenous particle administration, and before and after two applications of FUS to the rat brain show progressive decline in chemoconvulsant induced spike rates with 1 MHz FUS at the indicated estimated in situ pressure in 50 ms bursts, 1 Hz burst frequency.

FIGS. 7A and 7B illustrates in vivo drug delivery of the present invention.

FIG. 8A to 8C illustrates no tissue damage in the brain of mice having a drug delivered by a method of the present invention.

### DETAILED DESCRIPTION OF THE INVENTION

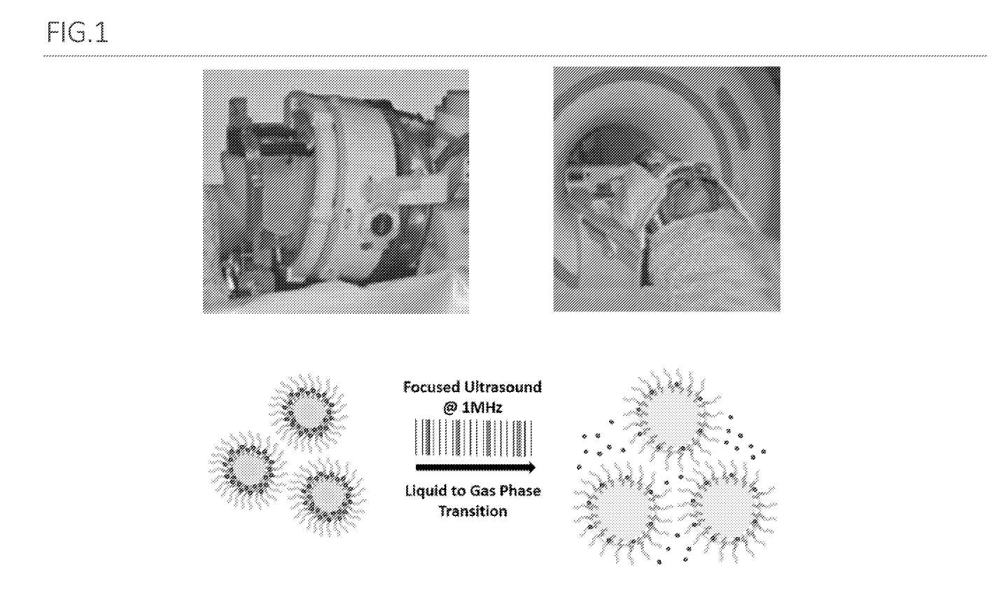

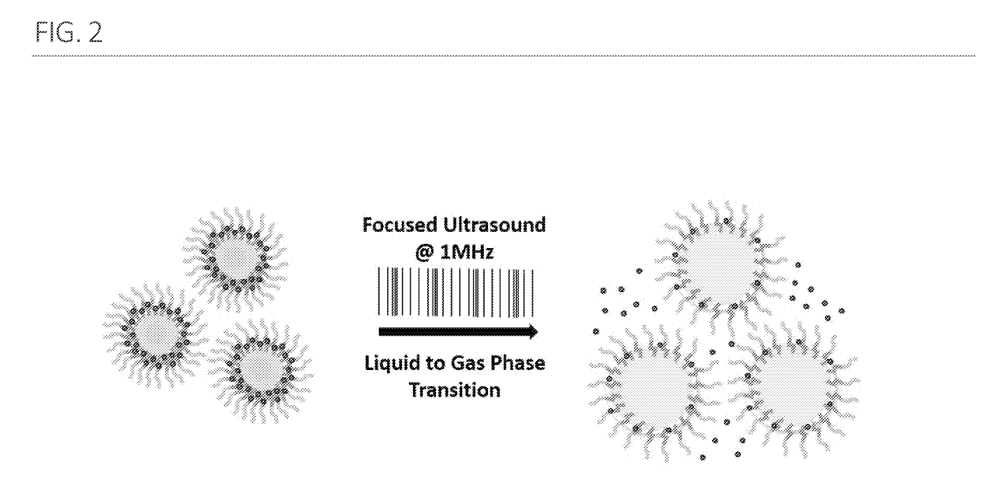

As shown in FIG. 1, the use of a clinical MR-guided Focused Ultrasound (MRgFUS) system was combined with nanoparticles that release a drug cargo upon sonication allow clinical neuromodulation that is noninvasive, image guided, regionally specific, safe and reversible, and the procedure may be performed while a subject is awake enabling communication with subject. Thereby accomplishing a goal of clinical and basic neuroscience that current strategies (TMS, DBS, tDCS, ECT, etc) fall short of. MRgFUS used in the present invention was shown to provide noninvasive, focal, and safe modulation of image-defined spatially compact regions of the brain at most any clinically interesting location. Specifically one embodiment of the present invention is a method of neuromodulating drug release in vivo using nanoparticles of the present invention and ultrasound. Nanoparticles of the present invention have been created to phase change in response to ultrasound, preferably provided by MRgFUS. The nanoparticles have a core containing a pharmaceutical agent, a composition that is initially in a liquid state but when the nanoparticles undergo ultrasound the composition turns into a gas exerting pressure on the walls of the nanoparticle, disrupting the walls of the nanoparticle and allowing the release of the pharmaceutical agent as shown in FIG. 2. Specifically, nanoparticles are preferably composed of a block copolymer (yellow and blue lines) encapsulating propofol (red dots) and a liquid perfluorocarbon droplet (light blue). Upon sonication, the liquid perfluorocarbon at the core of the particles transitions into a gas phase (lighter blue), triggering the release of a pharmaceutical agent, propofol, in this example.

The nanoparticles were prepared by making micelles of a polyester polymer (poly-caprolactone) and the drug or pharmaceutical agent (propofol), at a 10:1 polymer: drug w/w ratio in PBS. A liquid perfluorocarbon (perfluoropentane, PFP) was then added to a 4:1 PFP: polymer v/w ratio and the mixture was sonicated with an immersion 20 kHz tip sonicator at 30% max. intensity for 30 sec. The resultant mixture was then centrifugated (at 5 k rcf for 5 min) and resuspended twice to remove excess polymer and propofol. Residual propofol was removed by mixing with an equivalent volume of hexane and extracting to aqueous phase.

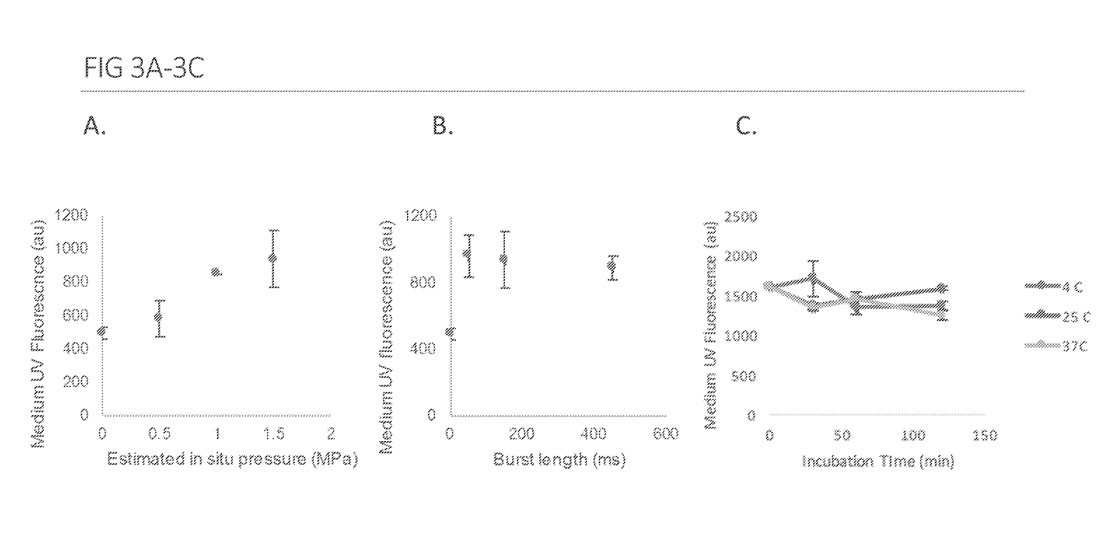

In vitro the particles were sonicated in a custom holder using a 1 MHz center frequency focused ultrasound transducer (RK300, FUS Instruments) with pulsed sonication (1 Hz burst frequency for 60 sec). Propofol release was assessed by extracting free propofol from the particles with hexane and assaying the UV fluorescence of the organic phase. The particles release propofol with a dose response with sonication pressure, but not burst length for the values tested, and are stable over the course of hours of incubation at varied temperatures as shown in FIG. 3. In vitro sonication-induced release of propofol versus in situ pressure (FIG. 3A) and burst length (FIG. 3B) demonstrates a dose response with in situ pressure but not with burst length. Propofol release was assessed by UV fluorescence of the organic medium after hexane extraction of free propofol, post sonication using pulsed focused sonication with 1 MHz transducer frequency with 1 Hz burst frequency for 60 sec (60 total bursts). Sonication burst length of 100 ms was used for FIG. 3A; and 1 MPa estimated in situ pressure was used for the FIG. 3B; n=3. The drug, or propofol in this example, was released when the internal pressure of the nanoparticle was in the range of 0.5 MPa to 2.0 MPa, 0.75 MPa to 2.0 MPa, 1.0 MPa to 2.0 MPa, 0.5 MPa to 1.5 MPa, or 1.0 MPa to 1.5 MPa.

### In-Vivo Validation

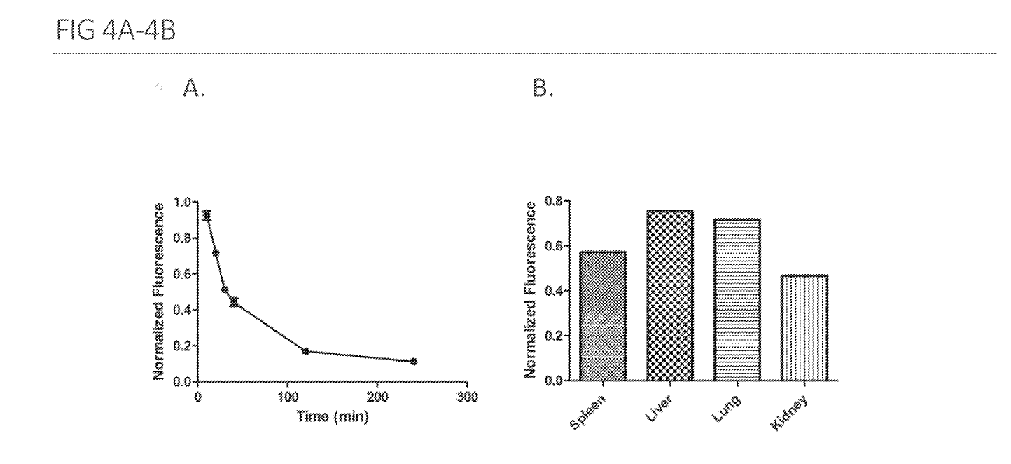

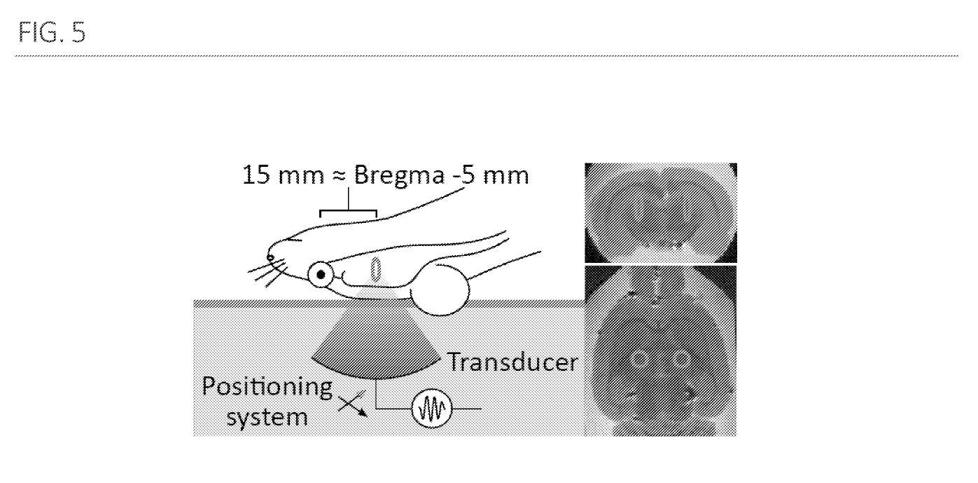

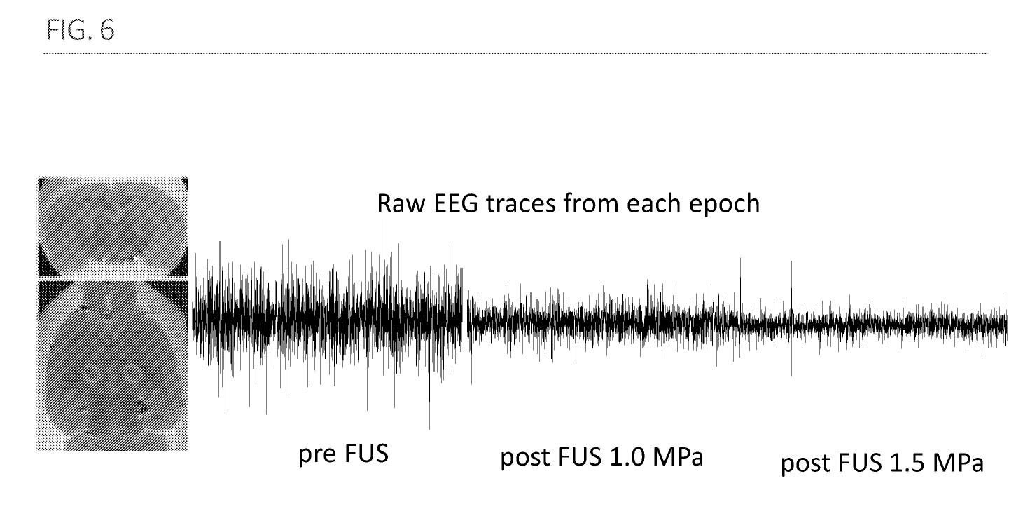

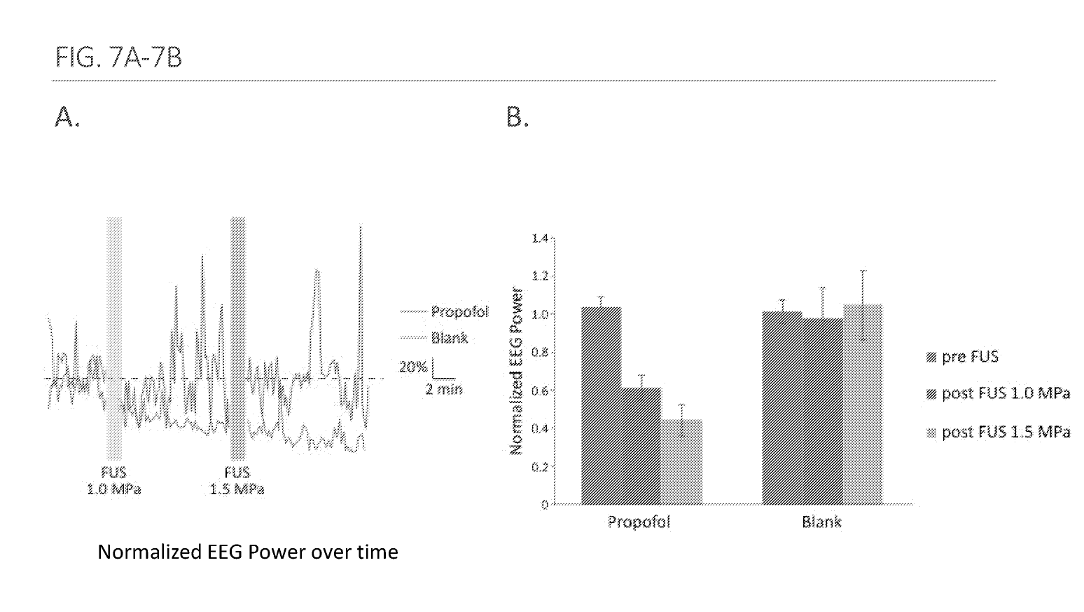

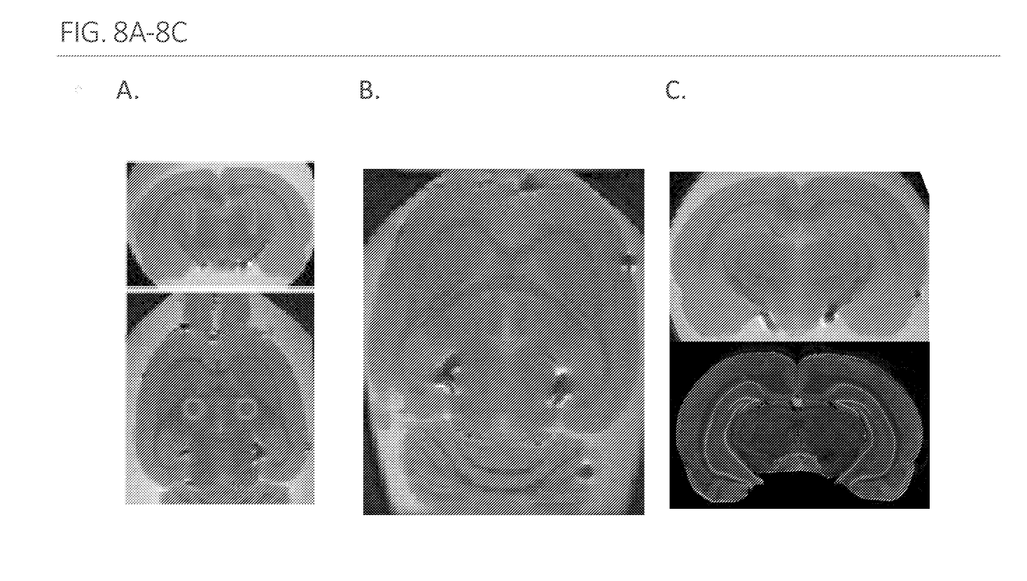

Nanoparticles of the present invention were doped with a custom infrared fluorescent dye (IR800, LICOR) and were administered via a 24 g tail vein catheter to rats (N=3) in a total volume of 1 cc (≈1 mg/kg propofol dose). Retro-orbital blood samples were taken over the course of 24 hours. As shown in FIG. 4A, at 24 hours, rats were euthanized and their organs harvested. Vascular dye fluorescence indicates an intravascular circulation half-life of ≈35 min (<2% of the initial amount was remnant at 24 h). As shown in FIG. 4B, nanoparticles were taken up in spleen, liver, lung, and kidney, with no substantial amount in the brain at 24 h. A rat seizure model was developed in which pentylenetetrazol (PTZ) was used to induce seizures, with the animal otherwise under ketamine/xylazine anesthesia. As shown in FIG. 5, after placing subdermal electrodes, rats were placed supine on the bed of a focused ultrasound transducer, with the transducer positioned with center 15 mm caudal to the center of the eyes, approximately 5 mm caudal to bregma per the Paxinos rat brain atlas. Following stable seizure induction, particles either loaded with propofol or no drug (‘Blank’) were administered to the rats via a tail vein catheter in 1 cc total volume (≈1 mg/kg propofol dose). Following >5 min baseline EEG acquisition, FUS was administered to two ˜1.5×5 mm foci, one in each hemisphere, in 50 ms bursts every 1 sec for 60 sec total, first at 1.0 MPa estimated in situ pressure, then at 1.5 MPa as illustrated in FIG. 6. Sample EEG traces after in vivo intravenous particle administration, and before and after two applications of FUS to the rat brain show progressive decline in chemoconvulsant induced spike rates with 1 MHz FUS at the indicated estimated in situ pressure in 50 ms bursts, 1 Hz burst frequency. For each trace, the total EEG power was calculated in 10 s bins, normalized to the preFUS baseline (average of 3 min prior to FUS), and averaged across the animals (N=7 propofol, 5 blank; two propofol animals had no seizure activity after the first FUS administration and did not receive FUS at 1.5 MPa). There were significant (p<0.05 for all comparisons) reductions of total EEG power with FUS for propofol treated animals but not for blank treated animals as shown in FIG. 7B. Following EEG, animals were euthanized by perfusion fixation and their brains were harvested. Ex vivo MRI was completed at 17.6 T (RARE, effective TE/TR 12.8/5000 ms, RARE factor 4; 0.16×0.16 mm pixels). Brains were then frozen and sliced on a cryotome, and then stained with cresyl violet for histological analysis. No evidence of tissue injury was identified as shown in FIG. 8. Consequently, the biodegradable nanoparticles, or drug carriers, permitted targeted, inducible release of drug cargo with focused ultrasound. The drug delivery using a method of the present invention is potent enough to interrupt and even halt seizure activity by the localized release of drug in the brain. There was no evidence of any deleterious consequence to the brain parenchyma of the particle administration or FUS application. The pre-sonication particle diameter was 400-450 nm. Upon sonication particles undergo a phase transition, increasing their diameter 5-6× and inducing drug release [3] (Figure, top), yielding a maximal particle diameter post-release of <3 μm, indicating no substantial risk of intravascular embolization. Focused ultrasound was sufficient to induce release of free propofol in vitro, with a dose response found with sonication pressure, but not with burst length (Figure, middle). During in vivo validation, spike rate decreases were seen following particle administration and focused ultrasound application indicating gated propofol release (Figure, bottom).

### Pharmaceutical Preparations

Pharmaceutical compositions of the present invention comprise an effective amount of one or more drug and a composition that undergoes a phase change from liquid to gas when ultrasound is applied within a nanoparticle of the present invention dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that comprises at least one additional active ingredient within the nanoparticles of the present invention will be known to those of skill in the art in light of the present disclosure, as exemplified by Remington: The Science and Practice of Pharmacy, 21^{st} Ed. Lippincott Williams and Wilkins, 2005, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient or nanoparticle, its use in the pharmaceutical compositions is contemplated.

The nanoparticle preferred routes of administration is injection. The nanoparticles may be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared in such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

### Kits of the Disclosure

Any of the compositions described herein may be comprised in a kit. In a non-limiting example, such compositions including a pharmaceutical agent, a material that expands when ultrasound in applied, and expandable polymer, may be comprised in a kit. Alternatively, nanoparticles of the present invention that comprise a pharmaceutical agent and a material that expands when ultrasound is applied may be part of a kit.

The kits may comprise a suitably aliquoted inducer of these compositions and, in some cases, one or more additional agents. The component(s) of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing one or more compositions and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred. One or more composition(s) or the nanoparticles of the present invention may be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means.

### EXAMPLES/METHODS

### Nanoparticle Formulation and Characterization

Micelles of polymer (50 mg; PEGylated poly-caprolactone, PEG-PCL) and propofol (5 mg) were made by dissolving each into 1 mL of anhydrous tetrohydrofuran (THF), then adding 1 mL of PBS, mixing, and then vacuum evaporation of the THF overnight. Micelles were then diluted 1:5 in PBS and perfluoropentane (PFP) was added to a net 1:4 polymer:PFP (w/v) ratio. To emulsify the PFP, the mixture was sonicated in 1 mL volumes with an immersion micro-tip sonicator operating at 20 kHz center frequency (Q500, QSonica, Newton, Conn.) operated at 30% maximum amplitude for 30 sec. Free polymer and propofol was then removed via centrifugation at 5,000 rcf for 5 min, then removal of the supernatant, and resuspension in fresh PBS. Centrifugation/resuspension was completed twice. Then mixture was then mixed with an equivalent volume of hexane to extract residual free propofol, and the aqueous phase of the mixture was collected for further experiments. Particle size was determined via dynamic light scattering with a ZetaSizer ( ) and via NanoSight ( ). For in vivo animal experiments, the above process was completed using sterile technique in cell culture hoods, with sterile reagents. For biodistribution experiments, 1 mg of a custom infrared fluorescent dye (IR800, LICOR Biosciences, Lincoln, Nebr.) was included in the original micelle mixture (50:1 polymer:dye ratio w/w).

To test particle release efficacy, the particles were sonicated by loading into a custom designed chamber sonicated using a focused ultrasound transducer (1 MHz center frequency; RK-300, FUS Instruments, Toronto, CA) with 10, 50, 100, or 150 ms bursts at 1 Hz burst frequency for 1 min (60 bursts) at either 0.5, 1.0, or 1.5 MPa in situ pressure. Released propofol was extracted via mixing the sonicated solution with hexane and extracting the organic phase. Propofol content in the organic phase was then quantified via assessing UV fluorescence at 280 ex/310 em on a plate reader ( ).

### Animals

All procedures included in this study were approved by the Johns Hopkins IACUC. Male Fischer 344 rats (150-200 gm weight) were used throughout these experiments. For biodistribution experiments, a tail vein cannula was placed while the animal was under isoflurane anesthesia (2% in oxygen supplied at 2 L/min). Animals were administered 1 mL of the sterile nanoparticle formulation, with a 100 μL sterile saline flush.

### Seizure Model, EEG Acquisition and Analysis

Rats were weighed and administered ketamine/xylazine (85/13 mg/kg) intraperitoneally for anesthesia. A tail vein cannula was placed. The dorsal fur was removed via electrical clipper and then a chemical depilatory (Veet, RB Inc, purchased through Amazon). This skin was then washed with saline and isopropanol. Four subdermal electrodes were placed with lead tips in the far lateral spaces, with two electrode tips anterior to bregma, and two leads near lambda. The lead wires were then connected to a headstage ( ) and placed to ensure that they did not cross the central dorsal scalp to allow for ultrasound transmission. The animal was placed supine on the bed of a focused ultrasound tranducer (1 MHz center frequency; RK300, FUS Instruments, Toronto, CA), with ultrasound gel used to couple the dorsal scalp to the animal bed, which was itself coupled to the ultrasound transducer with degassed water. The head orientation and position was fixed with a vendor provided bite bar and nose cone integrated with the transducer bed, via which supplemental oxygen was provided at 2 L/min. The headstage was then connected to the EEG acquisition system ( ).

Following acquisition of an EEG baseline of 5-10 min, animals were administered the chemoconvulsant pentylenetetrazole (PTZ) 45 mg/kg intraperitoneally. Animals were monitored via real-time EEG and visual inspection for evidence of convulsive and seizure activity. Repeat administration of 45 mg/kg doses of PTZ were administered until clear seizure activity was noted by both visual inspection and real-time EEG, within 5 min of the last PTZ dose. Animals required 2-4 doses of 45 mg/kg PTZ to achieve this state.

Animals were then administered the indicated sterile particles in 1 mL total volume intravenously with a 100 μL sterile saline flush. After several minutes to allow for stabilization of the EEG trace following any handling-related seizure activity and post-ictal depression, at least 5 min of a new EEG baseline was acquired. Focused ultrasound was then applied with 1.0 MPa estimated in situ pressure (estimated via the method of [ref Oreilly]) in 50 ms bursts delivered every 1 sec for a total of 1 min (60 bursts) delivered to each of two points 2.5 mm to the left and right of midline, 15 mm caudal to the eyes, which translates to approximately 5 mm caudal to bregma. 10 min of EEG traces were then acquired. Then, if convulsive/seizure activity persisted, FUS was applied as above except with 1.5 MPa of estimated in situ pressure. After 10 min more of EEG trace acquisition, an adequate depth of anesthesia was confirmed and the animal was euthanized via perfusion fixation or cervical dislocation. Perfused animals brains were then harvested. Throughout this procedure, ketamine/xylazine anesthesia depth was confirmed via toe pinch, and if toe pinch reflex was present then a repeat dose of ketamine/xylazine was given. However, if seizure induction with PTZ had been completed, and the animal was evidently waking from anesthesia, the animal was excluded from further experimentation.

For EEG analysis, EEG traces were first bandpass filtered and EEG power was calculated in 10 sec bins across each trace, calculated as total power and power within the theta band (6-10 Hz). Each power time course was normalized by its average power within the three minutes prior to particle administration.

### Ex Vivo MRI

Fixed brains harvested following EEG/FUS experiments were scanned while submerged in fixative on a 17.6 T MRI (Bruker 750 MHz) in axial and coronal planes using effective TE/TR=12.8/5000 ms, RARE factor=4. Matrix=128×128, FOV=20×20 mm.

### Histology

Following ex vivo MRI, fixed brains were transferred to a 15% sucrose for 3 days, then a 30% sucrose solution for 2 days and then frozen at −80 C. Brains were then sectioned in the coronal plane at 40 um thickness using a cryotome (Leica). Sectioned were allowed to dry at room temperature and then were stained with Cresyl Violet and imaged in bright field and with fluorescence.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A nanoparticle with a surface comprising an expandable polymer encasing a pharmaceutical composition comprising a pharmaceutical agent and a material that expands upon the application of ultrasound.

2. The nanoparticle of claim 1 wherein the material expands by transitioning from a liquid to gas.

3. The nanoparticle of claim 2, wherein the material is perfluoropentane.

4. The nanoparticle of claim 1, wherein the expandable polymer is a block copolymer.

5. The nanoparticle of claim 4, wherein the block copolymer is selected from the group consisting of PEGylated poly-caprolactone, PEGylated poly-L-lactide, or a combination thereof.

6. The nanoparticle of claim 1, wherein the pharmaceutical agent is a neuromodulatory agent.

7. The nanoparticle of claim 6, wherein the neuromodulatory agent is propofol.

8. A method of targeted release of a drug comprising the following steps:
a) administering to a subject a pharmaceutical composition comprising a nanoparticle with a surface comprising an expandable polymer encasing a pharmaceutical agent and a material that expands upon the application of ultrasound; and
b) applying ultrasound to an area of the subject adjacent to the nanoparticle so the material and surface expand forming an expanded surface that releases the pharmaceutical agent from the nanoparticle compared to when the ultrasound is not applied to the nanoparticle.

9. The method of claim 8 wherein the nanoparticle has a diameter and applying the ultrasound expands the diameter in the range of 5 to 6 times forming an expanded nanoparticle that releases pharmaceutical agent from the nanoparticle compared to when ultrasound is not applied to the nanoparticle.

10. The method of claim 8 where the material is a liquid that expands by turning into a gas.

11. The method of claim 8, wherein the expandable polymer is a block copolymer.

12. The method of claim 11, wherein the block copolymer is selected from the group consisting of PEGylated poly-caprolactone, PEGylated poly-L-lactide, or a combination thereof.

13. The method of claim 8, wherein the pharmaceutical agent is a neuromodulatory agent.

14. The method of claim 13, wherein the neuromodulatory agent is propofol.

15. The method of claim 8, wherein the ultrasound is applied with a tip sonicator.

16. The method of claim 8 where the ultrasound is applied at 20 kHz continuously in the range of 1 to 10 seconds.

17. The method of claim 8 wherein the ultrasound is applied with a focused ultrasound transducer.

18. The method of claim 8 wherein the ultrasound is applied at 1 MHz using 10 ms pulses every 1 sec for up to 2 min.

19. The method of claim 10 where the material is perfluoropentane.

20. A brain functional localization method comprising the step of:
a) administering to the brain of a subject a pharmaceutical composition comprising a nanoparticle with a surface comprising an expandable polymer encasing a neuromodulatory agent and a material that expands upon the application of ultrasound;
b) applying ultrasound to an area of the brain adjacent to the nanoparticle so the material and surface expand forming an expanded surface that releases the neuromodulatory agent from the nanoparticle compared to when the ultrasound is not applied to the nanoparticle.

21. The method of claim 20 wherein the nanoparticle has a diameter and applying the ultrasound expands the diameter in the range of 5 to 6 times forming an expanded nanoparticle that releases the neuromodulatory agent from the nanoparticle compared to when ultrasound is not applied to the nanoparticle.

22. The method of claim 20, wherein the material is a liquid that expands by turning into a gas.

23. The method of claim 20, wherein the polymer is a block copolymer.

24. The method of claim 20, wherein the neuromodulatory agent is propofol.

25. The method of claim 20, wherein the ultrasound is applied at 20 kHz continuously in the range of 1 to 10 seconds.

26. The method of claim 20 wherein the ultrasound is applied with a focused ultrasound transducer.

27. The method of claim 20 wherein the ultrasound is applied at 1 MHz using 10 ms pulses every 1 sec for up to 2 min.
